(19) United States
(12) Patent Application Publication
(10) Pub. No.: US 2007/0260104 A1
(43) Pub. Date: Nov. 8, 2007

(54) AUTOLOGOUS FAT PAD TRANSFER FOR USE WITH PARTIAL BREAST IRRADIATION

(76) Inventor: Phillip BRETZ, Rancho Mirage, CA (US)

Correspondence Address: DENNISON, SCHULTZ & MACDONALD, 1727 KING STREET, SUITE 105, ALEXANDRIA, VA, 22314, US

(21) Appl. No.: 11/745,791

(22) Filed: May 8, 2007

Related U.S. Application Data
(60) Provisional application No. 60/798,295, filed on May 8, 2006.

(51) Int. Cl. A61N 5/00

(52) U.S. Cl. 600/3; 600/1

(57) ABSTRACT

A method of treatment of breast cancer using a brachytherapy radiation source. A tumor is surgically removed from the breast, creating thereby a cavity, and an autologous fat pad, preferably an abdominal fat pad is transplanted under the surface of the skin within the cavity. A brachytherapy balloon catheter is then inserted through the skin of the patient into the cavity and inflated, and a radiation source is deposited into the balloon for treatment, with the fat pad creating a cushion of at least 7 mm, and preferably 10-12 mm, between the inflated balloon and the skin.

This application claims the benefit of provisional application 60/798,295, filed May 8, 2006.

### BACKGROUND OF THE INVENTION

1. Field of the Invention

The invention relates to the field of radiation treatment for breast cancer, particularly treatment using an implanted catheter to deliver high dose rate brachytherapy.

2. Description of Related Art

With the discovery of the famous Edwin Smith papyrus in 1862, we know the earliest recorded treatment of breast cancer dates back to 3000 BC to Imhotep, Egypt's premier physician who used a crude form of cautery to burn a tumor eroding through the skin.

Leonidus of Alexandria in the 1st century AD performed the first breast surgery involving cutting and LeDran in 1685 was the first to note the spread of breast cancer to the axillary lymph nodes. William Halstead, M.D. in 1894 published his radical mastectomy paper made possible by the discovery of nitrous oxide in 1846.

Halstead's rationale was based on the German pathologist, Rudolf Virchow, who felt that breast cancer spread by direct extension to the lymph nodes and that radical surgery could thus eliminate the disease and effect cure.

In more modern times, clinical trials were promulgated in the United States in the late 1950's by the National Surgical Adjuvant Breast Project (N.S.A.B.P.). From this group we learned about the systemic nature of breast cancer. The early study of Umberto Veronesi in 1979 and a 2002 follow up paper, Veronesi, Umberto, M.D., et al., Twenty year Follow up of a Randomized Study Comparing Breast-Conserving Surgery with Radical Mastectomy for Early Breast Cancer, The New England Journal of Medicine, Volume 347:1227-1232, Oct. 17, 2002, Number 16, confirmed that lumpectomy combined with external beam radiation yielded the same survival results as mastectomy, the treatment paradigm again shifted to investigate less invasive ways to treat breast cancer.

Giovanna Bonadana, From Adjuvant to Neo-adjuvant Chemotherapy in High Risk Breast Cancer: The Experience of the Milan Cancer Institute, International Journal of Cancer, 55(1):1-4 (Aug. 19, 1993), showed that invasive breast cancers could be pre-treated with chemotherapy before surgery, thus downstaging the tumor and permitting the surgeon to perform a lumpectomy rather then mastectomy.

In the late 1990's, Armando Giuliano helped sentinel lymph node biopsy into the armamentarium of mainstream surgeons.

The five paradigm shifting modalities in breast cancer treatment of the late 20th century including radiation, lumpectomy, neo-adjuvant chemotherapy, sentinel lymph node biopsy and Tamoxifen treatment for prevention have paved the way toward using partial breast irradiation with its potential benefits.

Since the seminal paper from Veronesi demonstrated the effectiveness of conservative breast surgery and external beam radiation vs. mastectomy, the treatment paradigm shifted to finding ever less invasive ways to treat breast cancer. Radiation treatment has typically consisted of six weeks of daily treatment, delivering 50-60 Gy, with possible complications like radiation pneumonitis.

Accelerated partial breast irradiation (APBI) is an alternative to conventional radiation in selected patients. The Mammosite method developed by Cytyc Corp. of Mountain View, Calif. uses an implanted, dual lumen balloon catheter shown in FIG. 1 to deliver high dose rate (HDR) brachytherapy, with a total dose of 34 Gy delivered to the portion of the breast at highest risk for recurrence in ten treatments over five days. While long term results with APBI are some years away, the MammoSite FDA Clinical Study of 2002 showed no local recurrences with median follow up of 29 months. To date, some 1,500 surgeons throughout the United States have been certified to use MammoSite. However, there appears to be a learning curve with regard to successful placement of the catheter, and a number of interdependent factors must be taken into account to achieve successful completion of partial breast irradiation. Among the important surgical considerations is maintaining a minimum of 7 mm of tissue between the surface of the balloon and the adjacent skin (so-called skin spacing interval). If this distance is not evident on CT scanning done before radiation commences, the balloon must be pulled.

### SUMMARY OF THE INVENTION

It is therefore an object of the invention to enable proper placement of the catheter to achieve successful partial breast irradiation.

To achieve this and other objects, the invention comprises a method of treating breast cancer including delivery of brachytherapy into the breast of a person in need thereof, comprising removing the tumor with creation of a cavity, and transplanting an autologous fat patch under the surface of the skin. When the brachytherapy catheter is inserted and the balloon inflated, the fat patch provides a cushion of at least 7 mm between the inflated balloon and the skin surface.

APBI is a new radiation concept based on supporting data that 90% of local breast cancer recurrences after lumpectomy take place within 1 cm from the lumpectomy site. This technique is very advantageous for infiltrating duct cell tumors less then 3 cm in diameter.

For patients who live away from a standard external beam radiation center, using the MammoSite method may mean the difference between a cosmetic lumpectomy vs. a mastectomy.

To optimize the radiation and cosmetic result, a patch having a thickness of 7-12 mm is suggested.

Refining the lumpectomy to incorporate a circumareolar incision and the autologous fat patch transfer makes it possible to obtain a superior cosmetic result and these techniques can be used to successfully achieve APBI.

### BRIEF DESCRIPTION OF THE DRAWINGS

FIG. 1 is a side view of a MammoSite® catheter being used for accelerated partial breast irradiation, representing the prior art;

FIG. 2a is an anterior view of a circumareolar incision according to the invention;

FIG. 2b is a lateral view of a circumareolar incision according to the invention; and

FIG. 3 is a lateral view of a fat pad secured to the undersurface of the skin forming a cushion.

### DESCRIPTION OF THE PREFERRED EMBODIMENTS

With the advent of an after loading balloon catheter for partial breast radiation following breast lumpectomy, the avenue has been opened to further the cosmetic result as well as continue the statistical significance of low recurrence and mortality. With the more recent advent of “in office placement” of the MammoSite® catheter, extreme care and attention to details must accompany each attempt at placement.

If the patient is a candidate for APBI, the surgeon at the time of lumpectomy must decide where to make the most advantageous incision both for cosmesis and obtaining the necessary 7 mm skin spacing. A circumareolar incision placed just inside the border of the areola is known to be an excellent method to excise breast tumors from any quadrant, but other methods of removing the tumor are also acceptable.

During the lumpectomy, the surgeon must sculpt the lumpectomy cavity to not only remove the tumor with clear margins but keep in mind the type of balloon, spherical vs. ellipsoid, and fashion the cavity accordingly. Assuming the surgeon will proceed with “in-office” placement of the MammoSite® catheter, a #22 Foley catheter is placed laterally through a stab wound while the patient is under anesthesia, keeping it parallel to the lumpectomy cavity. Because the trocar used to locate the MammoSite® catheter is large and making an incision in the office and pushing or leveraging the trocar thru the breast tissue to make the necessary tunnel can be painful, the Foley catheter is placed while the patient is under anesthesia, and only a substitution of the MammoSite catheter for the Foley catheter need take place while the patient is under local anesthesia in the office. It must also be kept in mind during the lumpectomy not to put any clips or other metal or plastic markers around the walls of the cavity to prevent those items from puncturing the balloon. Also, if the patient had a stereotactic core biopsy for diagnosis and an identification clip was placed, it is important to remove the clip at surgery using pre-op localization. This is important not only for the patient to get her full treatment, but also to protect the balloon.

If the lumpectomy is done on a Friday and the radiation starts the following Monday, this is adequate time for the Foley catheter to maintain the tunnel. Catheters can also be switched the day following the surgery without difficulty, depending on circumstances. The patient is provided with a sedative to take the morning of “in-office” placement and 1% lidocaine without epinephrine is used immediately before the exchange is made.

Local anesthesia can be avoided if the lumpectomy cavity is fresh and the surgeon knows the correct volume to fill the balloon to exactly configure the lumpectomy walls. The area is cleaned and the MammoSite® catheter is checked and filled and decompressed. Sterile Neosporin ointment is used to thinly cover the balloon and the catheter itself. With the Foley balloon decompressed, it is literally a ten second procedure to switch the Foley for the MammoSite® catheter. The MammoSite® balloon is then inflated and can be checked with ultrasound for accommodation to the lumpectomy walls. Two other important items are that the balloon will be filled with the approximate amount of fluid the surgeon used to fill the lumpectomy space during surgery, and the balloon need not be secured with a stitch, just a dry dressing. The patient is told to expect some drainage and making the stab incision during surgery wide enough allows fluid to egress so that no fluid or air is surrounding the balloon at CT done before treatment. The patients are covered with antibiotics starting just before surgery to the last day of treatment.

Surprisingly, the patient should experience no real pain with this procedure but will feel the pressure of the balloon. Pain may be experienced on inflation of the balloon if excessive time lags between the lumpectomy and the placement, and wound contracture makes inflation painful.

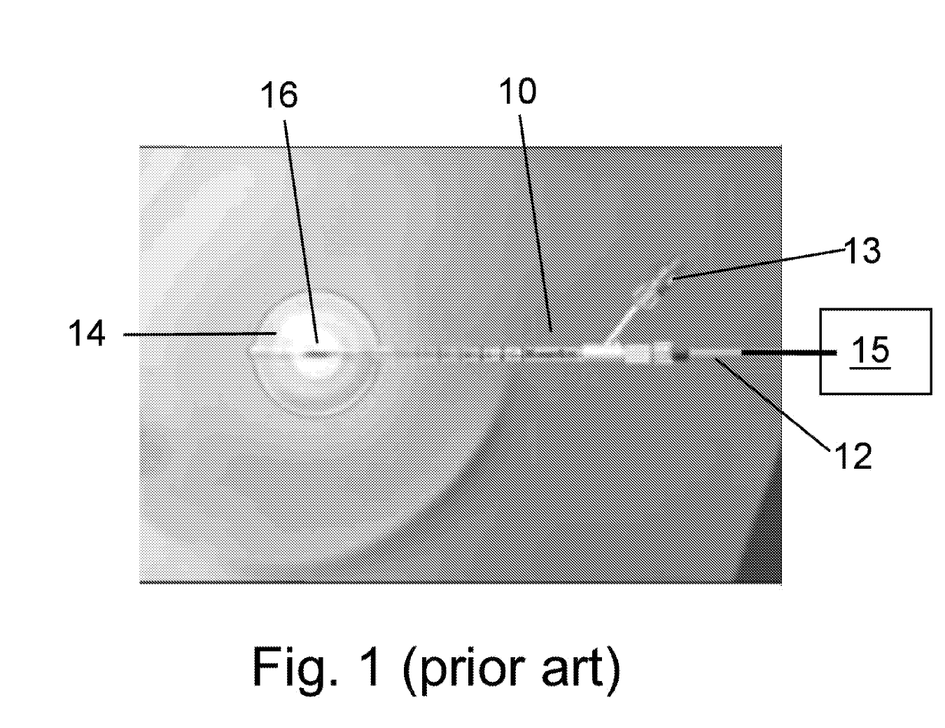

A MammoSite® catheter 10, as used in the prior art and according to the invention, shown in FIG. 1, is an after loading balloon catheter having a double lumen which allows inflating the balloon with contrast filled saline to the desired volume.

A stiff trocar in central lumen 12 is used to locate the catheter and balloon in the cavity, and the second lumen 13 is used to inflate balloon 14 surrounding the catheter with a saline contrast medium. A High Dose Radiation (HDR) iridium 192 radiation source 16 is moved through the catheter to the balloon end, where it remains for the time necessary for a treatment, utilizing a computer-controlled high-dose rate remote afterloader 15, and then removed. Treatments take place twice daily for five days, delivering 34 Gy in total. Currently there are multiple sized MammoSite® catheters with different fill volumes to choose from; depending on the configuration of the lumpectomy cavity, either a more rounded cavity or an oblong cavity may be selected.

One catheter choice is a round balloon as shown in FIG. 1, about 5 cm in length, which when optimally filled with fluid to 35-70 ml, occupies a round lumpectomy space. A newer balloon is the ellipsoid, about 8 cm in length, and occupies an oblong lumpectomy cavity. Both balloons are meant to exactly abut (be in contact with circumferentially) the margins of the lumpectomy site. The principal of the MammoSite® after loading balloon catheter is that about 90% of the local recurrences in the breast following lumpectomy are within 1 cm of the lumpectomy margin.

Three interdependent factors need to be in evidence for successful initiation and completion of partial breast irradiation using the MammoSite and they are:

(1) Adequate skin spacing,

(2) Good conformance and

(3) Good symmetry.

If one on these factors is not optimal then the balloon must either be repositioned or pulled.

Adequate skin spacing is a result of the surgeon configuring the lumpectomy cavity to maintain at minimum the 7 mm space between the margin of the balloon and the under surface of the adjacent skin.

Good conformance means that the balloon will conform circumferentially to the surrounding lumpectomy wall with ideally no air or fluid pockets.

Good symmetry means that the balloon is symmetrical, verified by CT.

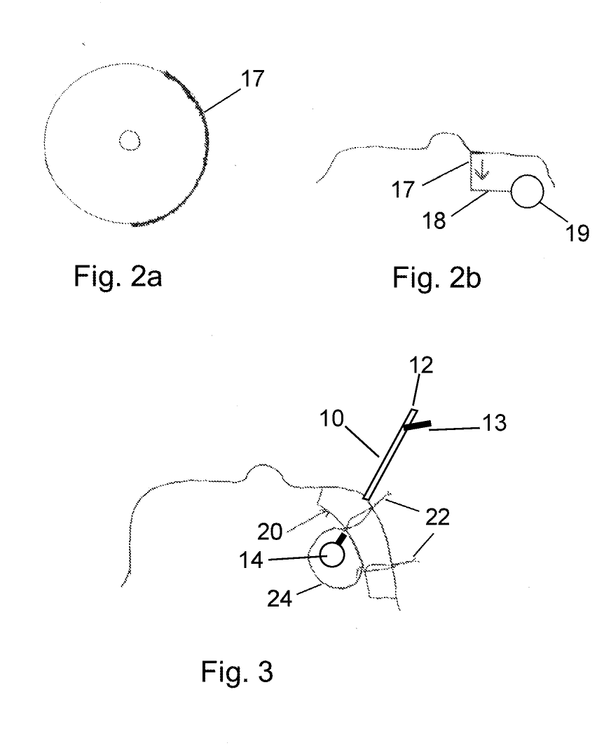

Performing a radial incision that just ellipses the tumor out as in excision of a melanoma makes it difficult to maintain the 7 mm skin spacing interval. Therefore, the invention utilizes a circumareolar incision 17 just inside the margin of the areola, as shown in FIGS. 2a and 2b, in which the surgeon intentionally goes straight down for a distance of about 1-2 cm and then tunnels 18 over to the site of the tumor 19. Depending on tumor location and size of the breast, adequate skin spacing can be maintained while the tumor is removed.

While excising the tumor and creating the lumpectomy cavity, the surgeon must keep in mind configuring the walls of the lumpectomy cavity so it will allow the balloon to conform to the lumpectomy walls over 360°.

This achieves a strong supportive wall/roof between the anterior surface of the lumpectomy cavity and the under surface of the skin. When optimally filled, the balloons of these catheters exert strong forces on the surrounding tissue so that the 7 mm skin spacing is absolutely necessary for the ultimate success of this endeavor. Skin spacing less then 7 mm may jeopardize the skin immediately anterior to the balloon. This one element of MammoSite® catheter placement has been a significant rate limiting step in that it may be decided during or before the lumpectomy that the needed skin spacing is not present. Also, after balloon placement, if the CT scan shows inadequate spacing, the balloon must be pulled. In either case, this may relegate the patient to a mastectomy if for any reason they cannot travel to and from the radiation center for six weeks of external beam radiation.

According to the invention, an autologous free fat patch transfer, preferably from the abdomen is used. The fat patch 20, shown in FIG. 3, is harvested a placed in the cavity through the incision after removal of the tumor; two tacking sutures 22 of 5-0 Monocril are used to stabilize the pad adjacent the cavity 24 left which is left after tumor removal, and the Foley catheter is then placed through the skin, adjacent the fat pad, into the cavity. The Foley catheter is replaced by the MammoSite® catheter 10 at the time that radiation treatment is to begin. The stay sutures are removed after the radiation treatment is completed and the catheter is removed.

If the breast is of sufficient size, the pad can simply be taken from an area about 180° from the lumpectomy site down low almost to the pectoral muscle. However, the abdominal pad is greatly preferred, in order to eliminate any possibility of tumor cells in the pad. The abdominal incision is made in the left lower quadrant so as not to confuse any physician examining the patient in the future that her appendix was removed, a rationale used for incision placement in the 1970's for servicemen returning from Vietnam who were being studied for exposure to Agent Orange. As the surgeon removes the patch, the 7 mm thickness should be kept in mind. After removal the patch is sculptured to exactly accommodate the lumpectomy site.

The autologous abdominal fat patch transfer has been used on nine consecutive patients to provide the adequate skin spacing. No adverse effects from the patch have been noted either on ongoing physical exam, ultrasound or mammography.

In a total of eighteen patients followed for up to twenty months, partial breast radiation has allowed a more rapid return to work without the universal tiredness associated with six weeks of external beam radiation. The cosmetic results are generally found to be excellent.

At this point, it is preferred that the patch be about 10-12 cm thick to avoid any skin problems.

The techniques presented here should allow the surgeon to place a MammoSite® catheter in the office setting without difficulty. Using the patch technique should help patients become candidates for APBI and avoid mastectomy or six weeks of external beam radiation and its potential complications.

The following are recommended steps for the method of the invention:

1. Anticipate the need for MammoSite® catheter treatment and discuss rational with patient.

2. Refer patient for pre-treatment planning session with oncologist and radiation therapist, scheduling the patient for APBI, CT and order catheter.

3. Plan the cosmetic lumpectomy on a Thursday, measure the fluid necessary to fill the lumpectomy space (that will be the balloon fill amount), including frozen section of margins and insert Foley or other suitable catheter through a lateral stab wound making sure there is enough space for egress of fluid. Switch the temporary catheter for the MammoSite® catheter on Friday.

4. Patient is covered with antibiotics throughout surgery and treatment.

5. Give patient a sedative (if deemed necessary) for that Friday.

6. Prepare catheter (before patient arrives) and patient in the usual sterile fashion.

7. Infiltrate the area of the lumpectomy walls with local anesthesia (optional).

8. Thinly coat the balloon and catheter with antibiotic ointment.

9. Switch the catheters when ready, making sure the Foley and the MammoSite® catheters are deflated.

10. Inflate the MammoSite® catheter. If pain is encountered, DO NOT use more local anesthetic; instead use ethyl chloride spray to cool the area and slowly inflate the balloon over several minutes. Switching the catheter the next day should obviate the need for local anesthesia and the balloon can be inflated rapidly.

11. After successful placement, patient is sent for CT scan before commencing APBI.

12. In order to hasten the process of in-office placement, it is suggested to prepare the MammoSite® catheter in total before the patient comes into the room so that less then five minutes passes between switching the Foley catheter for the MammoSite® catheter.

What is claimed is:

1. A method for treatment of breast cancer using a brachytherapy radiation source, comprising the steps of:
performing an incision in a breast and surgically removing a malignant tumor, creating thereby a cavity;
transplanting an autologous fat patch under the surface of the skin within the cavity;
inserting a distal end of a brachytherapy catheter having a balloon through the skin of the patient into the cavity; and
inflating the balloon, and depositing a radiation source into the distal end of the catheter for irradiation treatment, the fat patch establishing a cushion of at least 7 mm between the skin and the inflated balloon.

2. The method of claim 1, wherein the incision is a circumareolar incision, followed by tunneling straight down under subcutaneous tissue to the tumor, thereby creating a supportive wall.

3. The method of claim 1, wherein the fat pad has a thickness of about 7-12 mm.

4. The method of claim 1, wherein the balloon after inflation has a volume corresponding substantially to the volume of the cavity.

5. The method of claim 4, wherein the volume is about 35-70 ml.

6. The method of claim 1, additionally comprising placing a temporary catheter through the skin of the patient into in the cavity after the surgical removal of the tumor, wherein the inserting step comprises replacing the temporary catheter with the brachytherapy catheter.

7. The method of claim 1, wherein the fat pad is an abdominal fat pad.

8. The method of claim 1, additionally comprising holding the fat pad in place with sutures.

9. The method of claim 8, wherein the sutures are removed at the completion of the irradiation treatment.

10. In a method for treatment of breast cancer comprising a surgical lumpectomy to remove a tumor, creating thereby a cavity at the site of the tumor, followed by inserting a brachytherapy balloon catheter into the cavity, inflating the balloon and using the catheter to supply a brachytherapy radiation source for an irradiation treatment, the improvement comprising transplanting an autologous fat patch under the surface of the skin after the lumpectomy to create a cushion of at least 7 mm between the inflated balloon and the skin of the patient.

11. The method of claim 10, wherein the surgical lumpectomy is performed using a circumareolar incision in the skin of the patent and tunneling straight down under subcutaneous tissue, thereby creating a supportive wall.

12. The method of claim 10, wherein the fat pad has a thickness of about 7-12 mm.

13. The method of claim 10, wherein the balloon after inflation has a volume corresponding substantially to the volume of the cavity.

14. The method of claim 13, wherein the volume is about 35-70 ml.

15. The method of claim 10, additionally comprising placing a temporary catheter through the skin of the patient into the cavity after the surgical lumpectomy, wherein the inserting step comprises replacing the temporary catheter with the brachytherapy catheter.

16. The method of claim 10, wherein the fat pad is an abdominal fat pad.

17. The method of claim 9, additionally comprising holding the fat pad in place with sutures.

18. The method of claim 15, wherein the sutures are removed at the completion of the irradiation treatment.
